(19) United States
(12) Patent Application Publication
(10) Pub. No.: US 2025/0127563 A1
(43) Pub. Date: Apr. 24, 2025

(54) FLEXIBLE RADIOPAQUE CATHETER TIP THAT CAN WITHSTAND RF ENERGY

(71) Applicant: Boston Scientific Medical Device Limited, Galway (IE)

(72) Inventor: Maria Luk, Kleinburg (CA)

(21) Appl. No.: 18/917,718

(22) Filed: Oct. 16, 2024

Related U.S. Application Data
(60) Provisional application No. 63/591,714, filed on Oct. 19, 2023.

(51) Int. Cl. A61B 18/14; A61B 18/00; A61B 90/00

(52) U.S. Cl. CPC A61B 18/1492; A61B 90/39; A61B 2018/00077; A61B 2018/00083; A61B 2018/00101; A61B 2018/00184; A61B 2018/00363; A61B 2018/144; A61B 2018/1475; A61B 2090/3966

(57) ABSTRACT

A radiofrequency perforation system for accessing a location in or near a patient's heart is disclosed. The system includes a radiofrequency puncture wire having a wire proximal portion and a wire distal portion including an electrically active distal tip. The system further includes an elongated catheter defining a lumen adapted to receive and allow longitudinal translation of the radiofrequency puncture wire therein, the elongated catheter having a proximal portion and a distal portion, the proximal portion including a hub and the distal portion including a distal tip. The distal tip of the elongated catheter includes an inner catheter liner layer, an intermediate flexible heat shielding layer, and an outer radiopaque layer.

### CROSS REFERENCE TO RELATED APPLICATION

The present application claims priority to U.S. Provisional Patent Application No. 63/591,714, filed Oct. 19, 2023, the entire disclosure of which is incorporated herein by reference.

### TECHNICAL FIELD

The present invention relates generally to systems and methods usable to deliver energy within the body of a patient. More specifically, the present invention is concerned with an elongated catheter to better identify the precise location of a radiofrequency perforation device used for accessing a location in or near a patient's heart.

### BACKGROUND

Devices currently exist that use radiofrequency energy to create a puncture, channel, or perforation within a tissue located in a body of a patient. With these devices, it is crucial for a user to identify the tip of the radiofrequency perforation device that is puncturing the tissue located in the body of a patient. Generally, radiopaque metal bands, commonly platinum bands, are used in the distal portion of a catheter for visualization of the perforation device under fluoroscopy. Radiopaque platinum bands are thick and rigid and usually placed approximately 3-5 mm away from the catheter tip. These metal bands are not placed directly at the tip of the catheter due to consideration of other performance requirements. These performance requirements include, but are not limited to, tip atraumaticity, trackability and mechanical integrity. Thus, radiopaque metal bands used in the distal section of a catheter are not flexible, do not allow for the actual tip to be visualized, and may break off of the catheter resulting in a foreign object in the body of a patient.

Additionally, polymers with radiopaque fillers, for example tungsten filled PEBAX, provide a radiopaque solution for the catheter tip; however, these materials fail when exposed to high amounts of thermal energy. This thermal stress seen at the catheter tip may be as a result of delivering radiofrequency energy via a radiofrequency guidewire electrode that is at or near the electrode tip.

Against this background, there exists a continuing need in the industry to provide improved catheter tips for radiofrequency perforation systems and devices. An object of the present invention is therefore to provide such a system and device.

### SUMMARY

In Example 1, a radiofrequency perforation system for accessing a location in or near a patient's heart includes a radiofrequency puncture wire having a wire proximal portion and a wire distal portion including an electrically active distal tip. The radiofrequency perforation system further includes an elongated catheter defining a lumen adapted to receive and allow longitudinal translation of the radiofrequency puncture wire therein, the elongated catheter having a proximal portion and a distal portion, the proximal portion including a hub and the distal portion including a distal tip; wherein the distal tip of the elongated catheter includes an inner catheter liner layer, an intermediate flexible heat shielding layer, and an outer radiopaque layer.

Example 2 is the radiofrequency perforation system of Example 1 wherein the intermediate flexible heat shielding layer is reflowed onto the inner catheter layer.

Example 3 is the radiofrequency perforation system of any of Examples 1-2 wherein the outer radiopaque layer is reflowed onto the intermediate flexible heat shielding layer.

Example 4 is the radiofrequency perforation system of Example 1 wherein the outer radiopaque layer is a radiopaque metal coil.

Example 5 is the radiofrequency perforation system of Example 1 wherein the outer radiopaque layer is used to facilitate identification of the placement of the catheter within a patient.

Example 6 is the radiofrequency perforation system of Example 1 wherein the intermediate layer is used to produce flexibility to the catheter distal tip and wherein the intermediate layer includes heat shielding properties.

Example 7 is the radiofrequency perforation system of Example 1 wherein the inner catheter liner is made of PTFE.

Example 8 is the radiofrequency perforation system of Example 1 wherein the intermediate flexible heat shielding layer is made of a melt processable polymer.

Example 9 is the radiofrequency perforation system of any of Examples 1-8 wherein the intermediate flexible heat shielding layer is PEBAX.

Example 10 is the radiofrequency perforation system of Example 1 wherein the outer radiopaque layer is made of a radiopaque filled polymer.

Example 11 is the radiofrequency perforation system of any of Examples 1-10 wherein the outer radiopaque layer is tungsten filled PEBAX.

Example 12 is the radiofrequency perforation system of any of Examples 1-11 wherein the outer radiopaque layer is PEBAX.

Example 13 is the radiofrequency perforation system of Example 1 wherein the intermediate layer is between 0.001 inches to 0.003 inches in thickness.

Example 14 is the radiofrequency perforation system of Example 1 wherein the outer radiopaque layer is between 0.003 inches to 0.005 inches in thickness.

Example 15 is the radiofrequency perforation system of Example 1 the radiofrequency perforation system further comprising a dilator having a dilator body defining a dilator lumen and a tapered distal tip.

In Example 16, a radiofrequency perforation system for accessing a location in or near a patient's heart includes a radiofrequency puncture wire having a wire proximal portion and a wire distal portion including an electrically active distal tip. The radiofrequency perforation system further includes an elongated catheter defining a lumen adapted to receive and allow longitudinal translation of the radiofrequency puncture wire therein, the elongated catheter having a proximal portion and a distal portion, the proximal portion including a hub and the distal portion including a distal tip; wherein the distal tip of the elongated catheter includes an inner catheter liner layer, an intermediate flexible heat shielding layer, and an outer radiopaque layer.

Example 17 is the radiofrequency perforation system of Example 16 wherein the intermediate flexible heat shielding layer is reflowed onto the inner catheter layer.

Example 18 is the radiofrequency perforation system of Example 17 wherein the outer radiopaque layer is reflowed onto the intermediate flexible heat shielding layer.

Example 19 is the radiofrequency perforation system of Example 16 wherein the outer radiopaque layer is a radiopaque metal coil.

Example 20 is the radiofrequency perforation system of Example 16 wherein the inner catheter liner is made of PTFE.

Example 21 is the radiofrequency perforation system of Example 16 wherein the intermediate flexible heat shielding layer is made of a melt processable polymer.

Example 22 is the radiofrequency perforation system of Example 21 wherein the intermediate flexible heat shielding layer is PEBAX.

Example 23 is the radiofrequency perforation system of Example 16 wherein the outer radiopaque layer is made of a radiopaque filled polymer.

Example 24 is the radiofrequency perforation system of Example 23 wherein the outer radiopaque layer is tungsten filled PEBAX.

Example 25 is the radiofrequency perforation system of Example 16 the radiofrequency perforation system further comprising a dilator having a dilator body defining a dilator lumen and a tapered distal tip.

In Example 26, a radiofrequency perforation system for epicardial or transseptal access includes a dilator having a dilator body defining a dilator lumen and a tapered distal tip. The radiofrequency perforation system also includes a radiofrequency puncture wire having a wire proximal portion and a wire distal portion including an electrically active distal tip. The radiofrequency perforation system further includes an elongated catheter defining a lumen adapted to receive and allow longitudinal translation of the radiofrequency puncture wire therein, the elongated catheter having a proximal portion and a distal portion, the proximal portion including a hub and the distal portion including a distal tip; wherein the distal tip includes an inner catheter liner layer, an intermediate flexible heat shielding layer, and an outer radiopaque layer.

Example 27 is the radiofrequency perforation system of Example 26 wherein the intermediate flexible heat shielding layer is reflowed onto the inner catheter layer.

Example 28 is the radiofrequency perforation system of Example 27 wherein the outer radiopaque layer is reflowed onto the intermediate flexible heat shielding layer.

Example 29 is the radiofrequency perforation system of Example 26 wherein the outer radiopaque layer is a radiopaque metal coil.

Example 30 is the radiofrequency perforation system of Example 26 wherein the inner catheter liner is made of PTFE.

Example 31 is the radiofrequency perforation system of Example 26 wherein the intermediate flexible heat shielding layer is PEBAX.

Example 32 is the radiofrequency perforation system of Example 26 wherein the outer radiopaque layer is tungsten filled PEBAX.

In Example 33, a method of making a radiofrequency perforation system for epicardial or transseptal access includes providing a radiofrequency puncture wire having a wire proximal portion and a wire distal portion including an electrically active distal tip. The method of making a radiofrequency perforation system further includes advancing an elongated catheter defining a lumen adapted to receive and allow longitudinal translation of the radiofrequency puncture wire therein, the elongated catheter having a proximal portion and a distal portion, the proximal portion including a hub and the distal portion including a distal tip; wherein the distal tip includes an inner catheter liner layer, an intermediate flexible heat shielding layer, and an outer radiopaque layer.

Example 34 is the method of Example 33 wherein the intermediate flexible heat shielding layer is reflowed onto the inner catheter layer.

Example 35 is the method of Example 34 wherein the outer radiopaque layer is reflowed onto the intermediate flexible heat shielding layer.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A and 1B are schematic illustrations of a medical procedure within a patient's heart for gaining access to the transseptal and epicardial space, according to embodiments of the present disclosure.

FIG. 2 is a side view of a catheter with a radiofrequency puncture wire inserted therein used for medical procedures of FIGS. 1A and 1B, according to embodiments of the present disclosure.

FIGS. 3A-3B are partially sectioned and cross-sectional views of a distal portion of a catheter, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

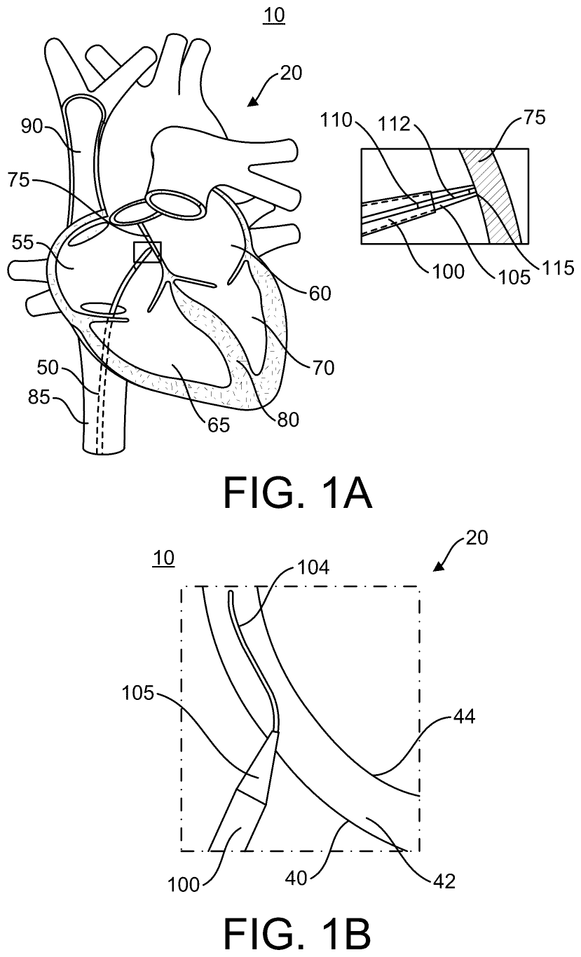

FIGS. 1A and 1B are schematic illustrations of a medical procedure within a patient's heart for gaining access to the transseptal and epicardial space, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a puncture device having distal end portion 112 terminating in a tip electrode 115, and a guidewire. In various embodiments, the puncture device 110 is a mechanical puncture device (e.g., a needle) or an RF perforation device. The puncture device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator may then be introduced into the sheath and over the guidewire, and advanced through the sheath into the SVC. Alternatively, the dilator may be fully inserted into the sheath prior to entering the body, and both may be advanced simultaneously towards the heart.

When the guidewire, sheath 100 and dilator 105 have been positioned in the SVC 90, the guidewire is removed from the body, and the sheath 100 and the dilator 105 are retracted so that their distal ends are positioned in the right atrium 55. The puncture device 110 described can then be introduced into the dilator 105, and advanced toward the heart 20. The puncture device 110 is then positioned such that the tip electrode 115 is aligned with or protruding slightly from the distal end of the dilator 105. In embodiments where the puncture device 110 is an RF perforation device, with the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

Still another medical procedure 10 developed for diagnosing or treating physiological ailments originating within a heart 20 includes epicardial ablation to help restore a regular heart rhythm, as shown in FIG. 1B. As illustrated, the heart 20 includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart 20 enclosed between the two opposing serosal surfaces, often referred to as the pericardial space or pericardial cavity 42.

In some embodiments, the pericardium 40 may be punctured with a puncture device 110, such as a needle (or other mechanical puncture device). Once punctured, a dilator 105 is advanced to dilate the puncture created by the needle through the pericardium 40. In certain embodiments, a sheath 100 may be advanced with the dilator 105 simultaneously. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel systems and methods for providing safe access to the heart, specifically transseptal and epicardial access, using radiofrequency energy. As will be explained in greater detail herein, the embodiments of the present disclosure improve the means of precisely locating a catheter tip and thereby a tip electrode of a radiofrequency puncture wire that is inserted into the body of a patient.

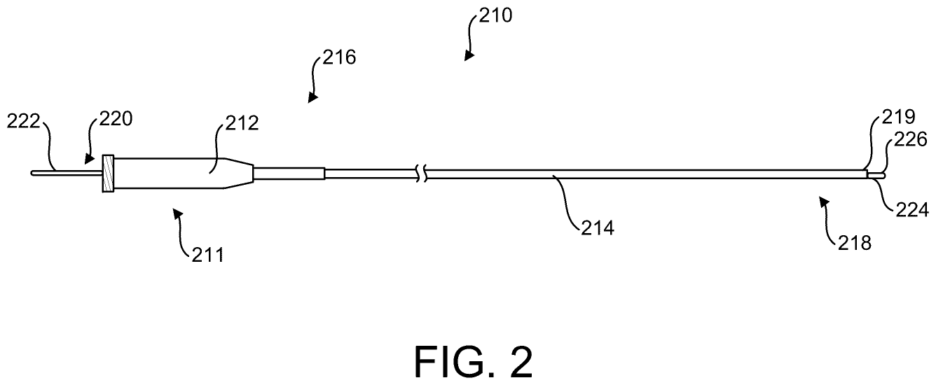

FIG. 2 is a side view of a catheter 210 with a radiofrequency puncture wire 220 inserted therein for accessing targeted regions in a patient's heart, according to embodiments of the present disclosure. As depicted, the RF puncture wire 220 includes a wire proximal portion 222 and a wire distal portion 224 terminating in an electrically active distal tip electrode 246 having radiofrequency energy emitted when activated. Additionally, as shown, the elongated catheter 210 defines a lumen (not shown) and includes a catheter proximal portion 216 and a catheter distal portion 218 extending from the proximal portion 216 and terminating at a distal tip 219. As shown, the catheter 210 further includes an elongate shaft 214 connected to a hub 211 having a main body portion 212. In some embodiments, the hub 211 is proximal to the elongate shaft 214. The catheter lumen extends longitudinally through the catheter 210 from the catheter proximal portion 216 to the catheter distal portion 218. In some embodiments, the elongate shaft 214 may preferably be a tubular member having a substantially uniform outer shape at the proximal end of the shaft 214. In other embodiments, the shaft 214 may further include a distal taper tapering at the catheter distal portion 218.

The catheter 210 serves to receive and allow a longitudinal translation of the RF puncture wire 220 inserted from the hub 211 and through to the catheter distal tip 219. In certain embodiments, the catheter 210 may be of a variety of different catheters used with a radiofrequency puncture wire including, but not limited to, balloon catheters, atherectomy catheters, drug delivery catheters, diagnostic catheters and guide catheters. Additionally, the catheter 210 may be sized in accordance with its intended use.

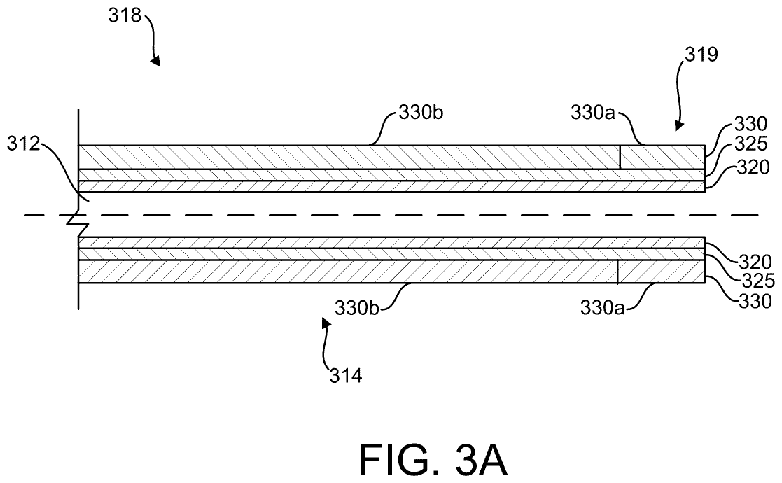

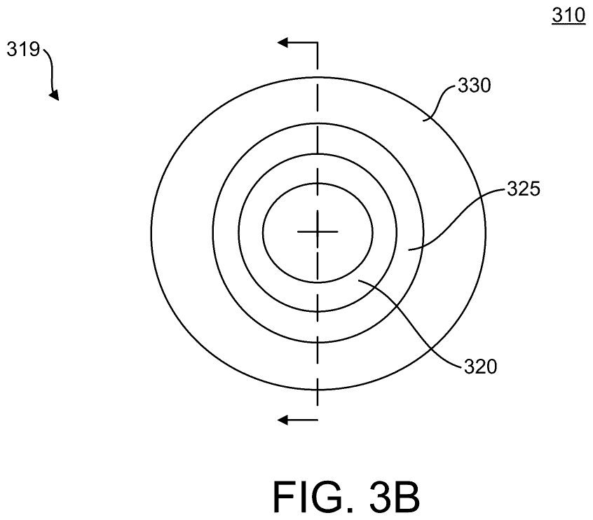

FIGS. 3A-3B are partially sectioned and cross-sectional views of an elongated catheter 310 having a catheter distal portion 318, according to embodiments of the present disclosure. The catheters of FIGS. 3A-3B are functionally and structurally similar to the catheter 210 of FIG. 2. The catheter 310 defines a lumen 312 extending longitudinally through the catheter 310 and more specifically through an elongated shaft 314. The elongated shaft 314 of the catheter 310 includes a catheter distal portion 318 terminating in a catheter distal tip 319. In some embodiments, the lumen 312 of the elongated shaft 314 allows a longitudinal translation of a RF puncture wire (not shown), with the RF puncture wire exiting through the catheter distal tip 319. As shown in FIG. 3A, the catheter distal portion 318 of the catheter 310 includes an inner catheter liner 320, an intermediate layer 325, and an outer radiopaque layer 330. The inner catheter liner 320 is closest to the lumen 312, while the outer radiopaque layer 330 is furthest from the lumen 312. Additionally, as shown, the outer radiopaque layer 330 includes a radiopaque filled polymer portion 330a and a polymer portion 330b. The radiopaque filled polymer portion 330a allows for trackability of the RF puncture wire as it is navigated in or near a patient's heart.

As shown, each of the inner catheter liner 320 and the intermediate layer 325 may extend from a proximal portion of the elongated shaft 314 to the distal tip 319. As further shown, in certain embodiments, the outer radiopaque layer 330 radiopaque filled polymer portion 330a may be placed at the distal tip portion 319 of the catheter to be able to better detect the distal tip portion 319 of the catheter. In certain embodiments, the outer radiopaque layer 330 radiopaque filled polymer portion 330a may be shorter or longer in length or spaced a certain distance from the catheter distal tip 319 for desired functionality.

In certain embodiments, the inner catheter liner 320 is made of polytetrafluoroethylene (PTFE). In some embodiments, the intermediate layer 325 is made of any melt processable polymer that can bond with a PTFE liner and the outer radiopaque polymer. One exemplary class of materials for construction of the intermediate layer 325 may include PEBAX. Another exemplary class of materials for construction of the intermediate layer 325 may include ceramic. In certain embodiments, the thickness of the intermediate layer 325 is between 0.001 inches to 0.003 inches. In other embodiments, the intermediate layer 325 is 0.001 inches in thickness. In certain embodiments, the intermediate layer 325 is flexible and may aid in shielding heat emitted from an activated tip of the RF puncture wire that is distal to the catheter distal tip 319. Thus, in one embodiment, the thicker the intermediate layer 325 of the distal tip 319, the more heat shielding properties the distal tip 319 of the catheter 310 may have.

In certain embodiments, the outer radiopaque layer 330 is made of any flexible radiopaque filled polymers that may bond to the intermediate layer 325. In certain embodiments, the distal portion of the outer radiopaque layer 330 is PEBAX mixed with tungsten in a range of from 50 to 90 percent by weight. In some embodiments, the radiopaque filled polymer portion 330a of the outer radiopaque layer 330 is made of 80 percent tungsten filled PEBAX. In certain embodiments, the polymer portion 330b of the outer radiopaque layer 330 is made of PEBAX. In certain embodiments, the thickness of the outer radiopaque layer 330 is between 0.003 inches to 0.005 inches. In other embodiments, the outer radiopaque layer 330 is 0.003 inches in thickness. The outer radiopaque layer 330 aids in visualization of the distal tip 319 of the catheter 310, and thus the distal portion of the RF puncture wire. Thus, the thicker the outer radiopaque layer 330, the more significant the visualization of the catheter may be under fluoroscopy.

Additionally, in some embodiments, the length of the outer radiopaque layer 330 may between 1 mm to 6 mm. In other embodiments, the length of the outer radiopaque layer 330 is 3 mm. Additionally, the longer the outer radiopaque layer 330 of the distal portion 318, the more visible the catheter distal portion 318 may be. In some embodiments, the distal tip 319 may include an inner catheter liner 320, an intermediate polymer layer 325, and a radiopaque metal coil as the outer radiopaque layer 330 for trackability. In certain embodiments, the radiopaque metal coil may be dense enough to provide adequate radiopacity, but not too dense to reduce flexibility of the distal tip 319.

Combining the inner catheter liner 320, the intermediate layer 325, and the outer radiopaque layer 330 creates multiple advantages to the catheter distal tip 319. In some embodiments, the combination of layers allows for the catheter distal tip 319 to be flexible. This flexibility allows the catheter to travel in or near the heart of a patient without causing any tissue damage. In certain embodiments, the combination of layers creates radiopacity without compromising the integrity of the catheter distal tip 319. More specifically, in some embodiments, the combination of the intermediate layer 325 with the outer radiopaque layer 330 allows the distal tip 319 of the catheter to withstand high amount of thermal energy that is emitted from an electrically active distal tip of an RF puncture wire without failing from heat degradation. Additionally, in certain embodiments, the outer radiopaque layer 330 may be sufficiently long in length without compromising trackability. In still other embodiments, by combining the PTFE inner catheter liner 320, the PEBAX filled intermediate layer 325, and the tungsten filled PEBAX outer radiopaque layer 330 at the catheter distal tip 319, a user may better and more efficiently visualize the catheter distal tip 319 under fluoroscopy and be able to make more precise perforations with a RF perforation device. This is more beneficial than having a radiopaque metal band at a distance away from the distal tip 319.

Furthermore, the inner catheter liner 320, intermediate layer 325, and outer radiopaque layer 330 are constructed at the catheter distal tip 319 using standard catheter assembly methods. This includes reflowing a thin layer of PEBAX onto the PTFE liner of the inner catheter liner 320. Then reflowing the outer radiopaque layer 330 onto the thin layer of PEBAX of the intermediate layer 325 and ensuring a strong mechanical bond.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A radiofrequency perforation system for accessing a location in or near a patient's heart, the system comprising:
a radiofrequency puncture wire having a wire proximal portion and a wire distal portion including an electrically active distal tip; and
an elongated catheter defining a lumen adapted to receive and allow longitudinal translation of the radiofrequency puncture wire therein, the elongated catheter having a proximal portion and a distal portion, the proximal portion including a hub and the distal portion including a distal tip;
wherein the distal tip includes an inner catheter liner layer, an intermediate flexible heat shielding layer, and an outer radiopaque layer.

2. The radiofrequency perforation system of claim 1, wherein the intermediate flexible heat shielding layer is reflowed onto the inner catheter layer.

3. The radiofrequency perforation system of claim 2, wherein the outer radiopaque layer is reflowed onto the intermediate flexible heat shielding layer.

4. The radiofrequency perforation system of claim 1, wherein the outer radiopaque layer is a radiopaque metal coil.

5. The radiofrequency perforation system of claim 1, wherein the inner catheter liner is made of PTFE.

6. The radiofrequency perforation system of claim 1, wherein the intermediate flexible heat shielding layer is made of a melt processable polymer.

7. The radiofrequency perforation system of claim 6, wherein the intermediate flexible heat shielding layer is PEBAX.

8. The radiofrequency perforation system of claim 1, wherein the outer radiopaque layer is made of a radiopaque filled polymer.

9. The radiofrequency perforation system of claim 8, wherein the outer radiopaque layer is tungsten filled PEBAX.

10. The radiofrequency perforation system of claim 1, the radiofrequency perforation system further comprising a dilator having a dilator body defining a dilator lumen and a tapered distal tip.

11. A radiofrequency perforation system for epicardial or transseptal access, the system comprising:
a dilator having a dilator body defining a dilator lumen and a tapered distal tip;
a radiofrequency puncture wire having a wire proximal portion and a wire distal portion including an electrically active distal tip; and
an elongated catheter defining a lumen adapted to receive and allow longitudinal translation of the radiofrequency puncture wire therein, the elongated catheter having a proximal portion and a distal portion, the proximal portion including a hub and the distal portion including a distal tip;
wherein the distal tip includes an inner catheter liner layer, an intermediate flexible heat shielding layer, and an outer radiopaque layer.

12. The radiofrequency perforation system of claim 11, wherein the intermediate flexible heat shielding layer is reflowed onto the inner catheter layer.

13. The radiofrequency perforation system of claim 12, wherein the outer radiopaque layer is reflowed onto the intermediate flexible heat shielding layer.

14. The radiofrequency perforation system of claim 11, wherein the outer radiopaque layer is a radiopaque metal coil.

15. The radiofrequency perforation system of claim 11, wherein the inner catheter liner is made of PTFE.

16. The radiofrequency perforation system of claim 11, wherein the intermediate flexible heat shielding layer is PEBAX.

17. The radiofrequency perforation system of claim 11, wherein the outer radiopaque layer is tungsten filled PEBAX.

18. A method of making a radiofrequency perforation system for epicardial or transseptal access, the method comprising:
providing a radiofrequency puncture wire having a wire proximal portion and a wire distal portion including an electrically active distal tip; and
advancing an elongated catheter defining a lumen adapted to receive and allow longitudinal translation of the radiofrequency puncture wire therein, the elongated catheter having a proximal portion and a distal portion, the proximal portion including a hub and the distal portion including a distal tip;
wherein the distal tip includes an inner catheter liner layer, an intermediate flexible heat shielding layer, and an outer radiopaque layer.

19. The method of claim 18, wherein the intermediate flexible heat shielding layer is reflowed onto the inner catheter layer.

20. The method of claim 19, wherein the outer radiopaque layer is reflowed onto the intermediate flexible heat shielding layer.
